(19) United States
(12) Patent Application Publication
(10) Pub. No.: US 2018/0185305 A1
(43) Pub. Date: Jul. 5, 2018

(54) An Amino Thiol for Use in the Treatment of an Infection Caused by the Bacterium Mycobacterium Spp

(71) Applicant: NOVABIOTICS LIMITED, Aberdeen (GB)

(72) Inventors: Deborah O'NEIL, Craibstone, Aberdeen (GB); Douglas FRASER-PITT, Craibstone, Aberdeen (GB)

(21) Appl. No.: 15/509,466

(22) PCT Filed: Jun. 6, 2016
(86) PCT No.: PCT/GB2016/051662

§ 371 Date: Mar. 21, 2018

Related U.S. Application Data
(60) Provisional application No. 62/173,615, filed on Jun. 10, 2015.

(30) Foreign Application Priority Data
Jun. 10, 2015 (GB) 1510077.9

(51) Int. Cl. A61K 31/145; A61K 9/00; A61K 45/06

(52) U.S. Cl. CPC A61K 31/145; A61K 45/06; A61K 9/0053

(57) ABSTRACT

The present invention relates to cysteamine, cystamine, or derivatives thereof, for use in the treatment and/or prevention of infection caused by the bacterium, Mycobacterium spp., in particular Mycobacterium abscessus, or a disease or condition associated therewith.

### CROSS-REFERENCE TO RELATED APPLICATIONS

This application is a U.S. National Phase of International Patent Application No. PCT/GB2016/051662, filed on Jun. 6, 2016 which claims priority to United Kingdom Application No. 1510077.9, filed on Jun. 10, 2015 and U.S. Provisional Application No. 62/173,615 filed on Jun. 10, 2015, the disclosures of which are incorporated herein by reference in their entirety for all purposes.

### FIELD OF THE INVENTION

The present invention relates to the use of cysteamine or cystamine, including derivatives thereof, in the treatment and/or prevention of infection caused by the bacterium, Mycobacterium spp., in particular Mycobacterium abscessus, or a disease or condition associated therewith.

### BACKGROUND TO THE INVENTION

Bacterial infections of mucus-rich environments such as the lung are common in diseases such as cystic fibrosis (CF). However, conventional antibiotics do not tend to work well in such environments and their antibacterial effectiveness is greatly diminished when used in such environments.

Cystic Fibrosis (CF) is an autosomal recessively inherited disease most prevalent in caucasian populations of European origin. In the UK there are about 10,000 people with CF, globally about 70-100,000 people are affected. The most important aspect of CF is respiratory, with the majority of CF associated morbidity and mortality being due to chronic suppurative lung disease and ultimately respiratory failure, currently median (95% CI) age of death in the UK is 29 (27-31). Bacterial infection and colonisation of the airways by a range of pathigens including emergening pathogenic species such as Mycobacterium abscessus is becoming more prevent and posing increasing clinical challenges. Mycobacteria Spp can not be treated successfully with existing therapies and preclude transplantation.

There remains a need for better therapies for treating and preventing these non-responsive/resistant bacterial infections, in particular those associated with mucous-rich environments such as the CF lung. In addition there remains a need to limit the amount or doses of antibiotics used with the introduction of alternative therapies or adjunct treatments that can improve the effectiveness of currently available treatments in the treatment or

### SUMMARY OF THE INVENTION

According to a first aspect of the invention there is provided cysteamine, cysteamine derivative, cystamine, cystamine derivative, or a pharmaceutically acceptable salt of any of the foregoing, for use in the treatment and/or prevention of infection caused by the bacterium, Mycobacterium spp., or a disease or condition associated therewith.

The inventors have found that cysteamine is surprisingly effective against Mycobacterium spp., in particular Mycobacterium abscessus.

In a further aspect, the invention provides an amino thiol, for example cysteamine, including derivatives thereof, for use in the treatment and/or prevention of infection caused by the bacterium, Mycobacterium spp., or a disease or condition associated therewith.

Preferably the Mycobacterium spp. is Mycobacterium abscessus.

Suitably, the infection may be a biofilm infection.

Suitably, the infection, or disease or condition associated therewith, may be selected from the group consisting of respiratory infections, infections in cystic fibrosis, skin and wound infections, middle-ear infections, gastrointestinal tract infections, peritoneal membrane infections, urogenital tract infections, oral soft tissue infections, formation of dental plaque, eye infections, endocarditis and infections of indwelling medical devices.

Suitably, cysteamine or cystamine, including derivatives thereof, may be used in a pharmaceutical composition comprising a pharmaceutically acceptable carrier, excipient or diluents. Thus the invention provides a pharmaceutical composition comprising cysteamine, and a pharmaceutically acceptable carrier or diluents, for use in the treatment and/or prevention of an infection caused by the bacterium, Mycobacterium spp., in particular Mycobacterium abscessus, or a disease or condition associated therewith.

Suitably, cysteamine or cystamine, including derivatives thereof, for use in accordance with the present invention may be used in combination with an antibiotic.

The invention further provides the use of cysteamine or cystamine, including derivatives thereof, in the manufacture of a medicament for the treatment and/or prevention of an infection caused by the bacterium, Mycobacterium spp., in particular Mycobacterium abscessus, or a disease or condition associated therewith.

In a further aspect, the present invention relate to a method of treating an infection caused by the bacterium, Mycobacterium spp., in particular Mycobacterium abscessus, or disease or condition therewith associated in a patient comprising administering an effective amount of cysteamine or cystamine, including derivatives thereof, to the patient.

In another aspect of the present invention, there is provided a method of preventing biofilm formation in an environment comprising the step of administering an effective amount of cysteamine or cystamine, including derivatives thereof, to the environment. Suitably, the environment may comprise the biofilm forming microorganism, Mycobacterium spp., in particular Mycobacterium abscessus.

Suitably, the environment may be the mouth.

Suitably, the microbial infection may be a systemic infection. Preferably, the systemic infection is a mucosal infection.

Suitably, the mucosal infection may be a gastrointestinal, urogenital or respiratory infection.

Suitably, the mucosal infection may be in a patient suffering from cystic fibrosis.

### BRIEF DESCRIPTION OF THE DRAWINGS

FIG. 1 is a histogram demonstrating the antimicrobial activity of tobramycin, cysteamine and combined tobramycin and cysteamine on polymicrobial load after 4 and 24 hours exposure. Bacterial load expressed as mean (95% confidence interval).

FIG. 2 is a histogram demonstrating the antimicrobial activity of ciprofloxacin, cysteamine and combined ciprofloxacin and cysteamine on polymicrobial load after 4 and 24 hours exposure. Bacterial load expressed as mean (95% confidence interval).

### DETAILED DESCRIPTION OF THE INVENTION

### Uses and Methods

The invention provides cysteamine or cystamine, including derivatives thereof, for use in the treatment and/or prevention of an infection caused by the bacterium, Mycobacterium spp., in particular Mycobacterium abscessus, or a disease or condition associated therewith.

In one embodiment the invention provides cysteamine or cystamine, including derivatives thereof, for use in the treatment of an infection caused by the bacterium, Mycobacterium spp., in particular Mycobacterium abscessus, in the lung of a cystic fibrosis patient.

The invention also provides a method of treating an infection caused by the bacterium, Mycobacterium spp., in particular Mycobacterium abscessus, or disease or condition therewith associated in a patient comprising administering an effective amount of cysteamine or cystamine, including derivatives thereof, to the patient.

The bacterial infection may typically be a disseminated infection or in particular be in a mucous-rich environment, such as the lung, for example the lung of a patient suffering from CF or bacterially-associated chronic obstructive pulmonary disease (COPD). The method of the present invention comprises the step of administering cysteamine or cystamine, including derivatives thereof, to the environment. The method may be in vivo or ex vivo.

The environment may comprise any bacterial infection, including an infection caused by more than one microorganism, for example bacteria and any one of fungi, yeast, viruses and protozoa.

The method or use of the invention may be used to minimise and, preferably, prevent the formation of bacterial colonies, in particular bacterial biofilms in a variety of environments including, but not limited to, household, workplace, laboratory, industrial environment, aquatic environment (e.g. pipeline systems), medical devices including indwelling devices such as defined herein, dental devices or dental implants, animal body for example human body.

The method or use of the invention may be used in the mouth to prevent the formation of plaque or caries on a human tooth or dental implant for example a denture.

The method or use of the invention may be used to prevent or restrict the formation of a bacterial colony especially a colony of Mycobacterium spp., in particular Mycobacterium abscessus. The method or use of the present invention may be used to prevent or treat bacterial infections including topical infections, oral infections and systemic infections. Topical infections may include wounds, ulcers and lesions for example, cutaneous wounds such cuts or burns, and conditions associated therewith.

Oral infections may include gingivitis, periodontitis and mucositis.

Systemic infections include infections associated with cystic fibrosis, COPD and other conditions associated with mucosal infections, for example, gastrointestinal, urogenital or other respiratory infections.

Examples of cysteamine derivatives include: 2-methylthio ethylamine (cinnamate), 2-methyl thio ethylurea, N-(2-methylthio ethyl) p-acetamido benzamide, 2-aminoethanethiol, N-(2-methylthio ethyl)p-acetamido benzenesulfonamide, N-(2-propylthioethyl)-p-methoxy benzamide, N-(butylthio ethyl) nicotinamide, N-(2-dodecylthio ethyl) p-butoxybenzamide, N-(2-methylthio ethyl) p-toluenesulfonamide, N-(2-isopropylthio ethyl) propionamide, N-(2-octylthio ethyl) acetamide, N-(2-butylthio ethyl) methanesulfonamide, N-(2-isopentylthio ethyl)butane, bis 1,4-(2-acetamido ethylthio), 2,3-butanediol, 2-hexadecylthio ethylamine hydrochloride, 2-allylthio ethylamine malate, 9-octadecene 2-ylthio ethylamine hydrochloride, 2-dodecylthio ethylamine hydrochloride, 2-isopentylthio ethylamine mandelate, 2-octadecylthio ethylamine salicylate, 2-β-hydroxyethyl thio ethylurea, 2-β-hydroxyethylthio ethylamine hydrochloride, 2-(2,3-dihydroxy propylthio)ethylamine p-toluenesulfonate, 2-(2-hydroxypropylthio)ethylamine oxalate, N-(2-methylthio ethyl)phenylacetamide, 2-(2,2-dimethoxy ethylthio) ethylamine hydrochloride, 2-(2,2-dimethoxy ethylthio) ethylamine undecylenate, 2-(2,2-diethoxy ethylthio) ethylamine undecylenate, 2-(2,2-diethoxy ethylthio)ethylamine acetate, 2-undecenylthio ethylamine, 2-β-ureidoethylthio ethylamine hydrochloride, 2-β-acetamidoethylthio ethylamine tropate, 2,2′-thio diethylamine fumarate, 2,2′-thio diethylurea, 3-β-aminoethylthio propylamine hydrochloride, S-β-ureidoethyl thiocarbamate, 2-ethoxycarbonylthio ethylamine hydrochloride, 2-dimethylamino carbonylthio ethylamine sulfate, 2-butoxycarbonyl methylthio ethylurea, 2-ethyloxycarbonylmethylthio ethylamine hydrochloride, 6-β-aminoethylthio hexanoate of methyl hydrochloride, 5-β-aminoethylthio pentanoic acid, 2-phenylthio ethylamine dihydrogen phosphate, 2-p-t-butylphenylthio ethylamine trichloroacetate, 2-p-methoxyphenylthio ethylamine ditartrate, 2-tolylthio ethylamine hydrobromide, 2-(1-biphenyl thio) ethylamine hydrochloride, 2-N-pentachlorophenylthio ethyl acetamide, 2-benzylthio ethylamine malate, 2-benzylthio ethylamine nicotinate, 2-benzylthio 2-methyl propylamine hydrochloride, 2-benzylthio propylamine lactate, N-(2-benzylthio ethyl)nicotinamide hydrochloride, N-(2-benzylthio ethyl) 10-undecene amide, N-(2-benzylthio ethyl) hexadecanamide, S-β-aminoethyl mercaptobutyric acid, N-(2-benzylthio ethyl)formamide, N-(2-benzylthio ethyl)phenylacetamide, N-[2-(2,6-dimethyl phenyl)ethyl]hexanamide, 2-o-aminophenylthio ethylamine succinate, N-(2-benzylthio ethyl) glutamine, S-β-aminoethyl mercapto acetic acid (3-S-β-aminoethyl) mercapto propionic acid, (3-S-.gamma.-amino propyl) mercapto acetic acid, S(2-p-methoxybenzamido ethyl) mercapto 2-(2-naphtyl methylthio) ethylamine hydrochloride, 2-(2-naphtyl methylthio) ethylamine disuccinate, (2-thenyl) 2-thio ethylamine hydrobromide, 2-N-acetyl (2-thenylthioethylamine, 2-o-chlorobenzylthio ethylamine hydrochloride, 2-p-chlorobenzylthio ethylamine glycolate, 2-o-fluorobenzylthio ethylamine hydrochloride, 2-furfurylthio ethylamine hydrochloride, 2-tetrahydrofurfurylthio ethylamine p-amino-benzoate, 2-β-phenylethylthio ethylamine glutamate, 2-diphenylmethylthio ethylamine hydrochloride, 2-triphenyl methylthio ethylamine hydrochloride hemihydrate, 2-(2-pyridyl ethylthio)ethylamine hydrochloride, 2-(2-p-toluene sulfonamido ethylthio) pyridine N-oxide, 2-β-aminoethylthiomethyl pyridine N-oxide dihydrochloride, 2-β-aminoethylthio pyridine N-oxide hydrochloride, 2,4-dichloro 2-benzylthio ethylamine aspartate, N-[2-(3,4-dichloro benzylthio)ethyl] butyramide, N-[2-(2,6-dichloro benzylthio)ethyl] dodecanamide, N-[2-(3,5-dichloro benzylthio)ethyl] trifluoroacetamide hydrochloride, 2-p-ethoxybenzylthio ethylamine hydrochloride, N-[2-m-fluorobenzylthio ethyl] chloroacetamide, 2-p-bromobenzylthio ethylamine succinate, 2-(3,4-dimethoxy benzylthio)ethylamine malate, 2-(3,4-methylenedioxy benzylthio)ethylamine hydrochloride, 2-(2,4-dichloro cetylthio)ethylamine, 2 (3,4,5-trimethoxy benzylthio)ethylamine hydrocinnamate, 2-p-methoxy benzylthio ethylamine salicylate, 2-o-methylbenzylthio ethylamine phenyl-acetate, N-[2-p-dimethylaminobenzylthio ethyl] methane-sulfonamide, 2-p-phenoxybenzylthio ethylamine hydrochloride, 2-β-aminoethylthio pyridine hydrochloride, 2-benzylthio ethylamine citrate, N-[2-benzylthio ethyl] 2,4-dihydroxy 3,3-dimethyl butyramide, N-(2-benzylthio ethyl) 6,8-dihydroxy 7,7-dimethyl 5-oxo 4-aza octanamide, N-[2-(2-pyridyl thio)ethyl] propionamide, 2-(2-pyridyl methylthio)ethylamine dihydrochloride, 2-benzylthio ethylamine pantothenate, S-(β-acetamidoethyl)mercaptoacetate of β-morpholinoethyl, S-(β-phenylacetamidoethyl)mercaptoacetate N′-methyl 2-piperazino ethyl, S-(3-ureidoethyl)mercaptoacetate of β-pyrrolidino-ethy, S-(β-trifluoroacetamidoethyl)-β-mercapto-propionate of β-dimethylaminoethyl, 2-p-nitrobenzylthio ethylamine crotonate, 2-β-morpholinocarbonyl ethylthio ethylamine hydrochloride, N,N-di(hydroxyethyl)S-(β-benzamido-ethyl) mercapto-acetamido, N[2-N′-methyl piperazino carbonylthio ethyl]acetamide, 2-(1-naphthyl thio)ethylamine hydrochloride, N-(3-β-ureidoethylthio propyl) succinamic acid, 3-allylthio propylamine, 3-(2,2′-dimethoxy ethylthio)propylamine, 3-(2,2′-dimethoxy ethylthio)propylamine sulfate, S-β-aminoethylmercapto acetic acid, the hydrochloride of S-β-aminoethyl mercapto acetic acid, N-(2-benzylthioethyl)acetamide, N-(2-benzylthioethyl)propionamide, N-(2-benzylthioethyl)butyramide, N-(2-benzylthioethyl)methanesulfonamide, N-(2-benzylthioethyl)ethanesulfonamide, N-(2-benzylthioethyl-propanesulfonamide, N-(2-benzylthioethyl)butanesulfonamide, S-(2-p-acetamidobenzenesulfonamido ethyl) mercapto acetic acid, S-(2-p-acetamidobenzamido ethyl) mercapto acetic acid, N-(2-thenylthioethyl)acetamide, 2-benzylthio propylamine, 2-benzylthio 2-methyl propylamine, 2-(2-p-toluenesulfonamido ethylthio) pyridine N-oxide, S-(2-p-butoxybenzamidoethyl)mercapto acetic acid, 2-t-butylthio ethylamine hydrochloride, 2-methoxycarbonyl methylthio ethylamine hydrochloride, 2-ethoxycarbonylmethylthio ethylamine hydrochloride, 2-propoxycarbonylmethyl thio ethylamine hydrochloride, 2-butoxycarbonylmethylthio ethylamine hydrochloride, 2,2′-thio diethylamine dihydrochloride, 3-(2-aminoethylthio)alanine hydrochloride, 2-benzylthio ethylammonium diacid phosphate, 2-methylthio ethylamine, N-(methylthioethyl) p-acetamidobenzamide, N-(2-methylthioethyl)nicotinamide, N-(2-methylthioethyl)benzamide, N-(2-methylthioethyl) p-butoxybenzamide, N-(2-methylthioethyl) butyramide, N-(2-methylthioethyl) propionamide, N-(2-methylthioethyl) acetamide, N-(2-methylthioethyl) butanesulfonamide, N-(2-octylthioethyl) methanesulfonamide, 2-cetylthio ethylamine hydrochloride, 2-(2-hydroxyethylthio) ethylamine hydrochloride, 2-methylthio ethylamine phenylacetatesnd 2-methylthio ethylamine undecylenate

By an “effective” amount or “therapeutically effective amount” is meant an amount of one or more active substances which, within the scope of sound medical judgment, is sufficient to provide a desired effect without excessive toxicity, irritation, allergic response, or other problem or complication, commensurate with a reasonable benefit/risk ratio.

Suitably the method or use of the invention is in the prevention of, delay of progression of, or treatment of a disease or condition selected from the group consisting of skin and wound infections, middle-ear infections, gastrointestinal tract infections, peritoneal membrane infections, urogenital tract infections, oral soft tissue infections, formation of dental plaque, eye infections (including contact lense contamination), endocarditis, infections in cystic fibrosis, and infections of indwelling medical devices such as described herein.

### Products of the Invention

In accordance with the present invention, cysteamine or cystamine, including derivatives thereof, may optionally be used in combination with an antibiotic. Thus the present invention provides a product comprising cysteamine or cystamine, including derivatives thereof, and an antibiotic agent.

The term “antibiotic” is used to refer to antibacterial agents that may be derived from bacterial sources. Antibiotic agents may be bactericidal and/or bacteriostatic.

Generally the antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracyclins. Alternatively or additionally the antibiotic agent may be effective against mycobacteria.

According to one embodiment, the antibiotic agent may be an aminoglycoside such as Amikacin, Gentamicin, Kanamycin, Neomycin, Netilmicin, Tobramycin or Paromomycin. In one embodiment, the antibiotic is amikacin or tobramycin. In another embodiment, the antibiotic is amikacin. Preferably, tobramycin.

According to one embodiment, the antibiotic agent may be an ansamycin antibiotic such as Geldanamycin and Herbimycin

Alternatively the antibiotic agent may be a carbacephem such as Loracarbef.

According to a further embodiment, the antibiotic agent is a carbapenem such as Ertapenem, Doripenem, Imipenem/Cilastatin or Meropenem.

Alternatively the antibiotic agent may be a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. Alternatively the antibiotic agent may be a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime and Ceftobiprole.

The antibiotic agent may be a lincosamides such as Clindamycin and Azithromycin, or a macrolide such as Azithromycin, Clarithromycin, Dirithromycin, Erythromycin, Roxithromycin, Troleandomycin, Telithromycin and Spectinomycin.

Alternatively the antibiotic agent may be a monobactams such as Aztreonam, or a nitrofuran such as Furazolidone or Nitrofurantoin.

The antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

The antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX).

The antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

According to one embodiment, the antibiotic agent may be a polypeptide such as Bacitracin, Colistin and Polymyxin B.

Alternatively, the antibiotic agent may be a tetracycline such as Demeclocycline, Doxycycline, Minocycline and Oxytetracycline

Alternatively or additionally the antibiotic agent may be effective against mycobacteria. In particular the antibiotic agent may be Clofazimine, Lamprene, Dapsone, Capreomycin, Cycloserine, Ethambutol, Ethionamide, Isoniazid, Pyrazinamide, Rifampicin, Rifabutin, Rifapentine or Streptomycin.

In one embodiment, the antibiotic agent is selected from tobramycin, ciproflaxin, meropenem, amikacin and azithromycin.

Generally the antibiotic agent is active in the treatment or prophylaxis of infections caused by gram-negative or gram-positive bacteria, such as Escherichia coli and Klebsiella particularly Pseudomonas aeruginosa.

The ratio of cysteamine or cystamine, including derivatives thereof, to antibiotic in the products of the invention may be from 1:10 to 10:1; generally at least 2:1 for example at least 3:1 or 4:1. Alternatively, the ratio of the antibiotic to cysteamine in the products of the invention may be from 1:100 1:2000, for example from 1:500 to 1:1000. According to one embodiment, the ratio of the antibiotic agent to cysteamine is approximately 1:1. Preferably the antibiotic is a non-peptide antibiotic and the ratio from 2:1 up to 4:1 to cysteamine. According to a further embodiment the ratio may be approximately 1:1.

More than Additive/Synergistic Effect

Surprisingly, it has been found that the antibacterial action of the antibiotic agent and cysteamine is more than additive, and generally increase synergistically, upon combination.

The Fractional Inhibitory Concentration (FIC) corresponds to an interaction coefficient indicating whether the combination of antimicrobial agents is synergistic, additive, antagonist or neutral. The FIC is determined by comparing the activity of an agent in combination (MIC of agent A+agent B) with the activity of the agent alone (MIC of agent A or agent B) as follow (Singh et al., 2000):

FIC=MIC_{A[combition]}/MIC_{A[alone]}+MIC_{B[combination]}/MIC_{B[alone]}

Additive combinations of two antimicrobial agents are indicated by a FIC index of 1, whereas a FIC index <1 indicates synergistic combinations. Neutral combinations would give a FIC between 1 and 4, a FIC index higher than 4 indicates antagonist effects between the two antimicrobial agents.

The FIC index of the combination of the components of the product of the present invention may be less than 1, typically less than 0.9, suitably less than 0.8, advantageously less than or around 0.75, for example less than or around 0.5. Alternatively, the FIC index of the combination of the components of the product of the present invention may be more than 1; generally between 1 and 2; typically between 1 and 1.5, suitably between 1 and 1.2.

The antibiotic agent and cysteamine may act synergistically and when administered together or sequentially the antibacterial activity of the active agents is far higher than when administered separately.

It is believed that the effect of cysteamine, upon co-administration or combination, with an antibiotic agent, is to reverse the resistance of the Mycobacterium spp. to said antibiotic agent. In other words cysteamine overcomes the insensitivity of the Mycobacterium spp. to the antibiotic. The result is surprising and could not have been predicted.

Generally, the antibacterial activity of the product of the present invention is at least two times greater than the antibacterial activity of antibiotic agent alone, typically the antibacterial activity of the product of the present invention is at least four times higher than the antibiotic agent alone, suitably at least an eight times higher, generally at least, or around ten times higher.

Generally the minimal inhibitory concentration (MIC) of the product of the present invention is at least two times lower than the MIC of the antibiotic agent alone in connection with the same bacterial pathogen, suitably at least four times lower, typically at least eight times lower, advantageously at least or around ten times lower.

To obtain synergistic effect the agents of the product of the present invention may be administered together or sequentially, preferably no more than 10 minutes apart.

The products of the invention may include synergistically effective amounts of each active agent defined herein. The invention therefore includes products comprising a synergistically effective amount of (i) cysteamine and (ii) an antibiotic agent which is different from (i). The product may be for use in the manufacture of a medicament, for simultaneous, separate or sequential administration for the treatment and/or prevention of an infection caused by the bacterium, Mycobacterium spp., in particular Mycobacterium abscessus, or a disease or condition associated therewith. The infection may be a biofilm infection. “Synergistically”, as used herein, may describe the action of the two or more active agents of the product of the invention working together to produce an effect greater than the expected combined effect of the agents used separately.

### Mode of Administration

The active agents may be administered simultaneously, sequentially or separately. The active agents may be provided as a combination package. The combination package may contain the product of the invention together with instructions for simultaneous, separate or sequential administration of each of the active agents. For sequential administration, the active agents can be administered in any order.

The term “active agent” is used to refer to cysteamine or cystamine, including derivatives thereof, and/or an antibiotic agent.

The active agents mentioned in this specification can exist in different forms, such as free acids, free bases, esters and other prodrugs, salts and tautomers, for example, and the invention includes all variant forms of the agents.

The active agent(s) of the product of the invention may be provided as pharmaceutical compositions additionally containing one or more pharmaceutically acceptable diluents, excipients and/or carriers. This applies to both fixed and free combinations.

The active agent(s) of the present invention may be administered by any suitable route known to those skilled in the art, preferably in the form of a pharmaceutical composition adapted to such a route, and in a dose effective for the treatment intended. The active agent(s) and composition may, for example, be administered parenterally, orally, intranasal, intrabronchial, enterally, transdermally, sublingually, rectally, vaginally, ocularly, or topically. Both local and systemic administration is contemplated.

For the purposes of parenteral administration (“parenteral” as used herein, refers to modes of administration which include intravenous, intramuscular, enteral, intraperitoneal, intrasternal, subcutaneous and intraarticular injection and infusion of which intravenous (including continuous intravenous administration) is most preferred) solutions in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

The active agent(s) or products of the invention can also be administered intranasally or by inhalation and are conveniently delivered in the form of a dry powder inhaler or an aerosol spray presentation from a pressurised container, pump, spray, atomiser, nebuliser, with or without the use of a suitable propellant.

Alternatively, the active agent(s) or the products of the invention can be administered in the form of a suppository or pessary, or they may be applied topically in the form of a gel, hydrogel, lotion, solution, cream, ointment or powder. The active agent(s) or products of the invention may be dermally or transdermally administered, for example, by use of a skin patch, depot or subcutaneous injection. They may also be administered by pulmonary or rectal routes.

For oral administration, the active agent(s) or product/composition of the invention may be in the form of; for example, a tablet, capsule, suspension or liquid. The composition is preferably made in the form of a dosage unit containing a particular amount of the active ingredient. Examples of such dosage units are capsules, tablets, powders, granules or a suspension, with conventional additives such as lactose; mannitol, corn starch or potato starch; with binders such as crystalline cellulose, cellulose derivatives, acacia, corn starch or gelatins; with disintegrators such as corn starch, potato starch or sodium carboxymethylcellulose; and with lubricants such as talc or magnesium stearate. The active ingredient may also be administered by injection as a composition wherein, for example, saline, dextrose or water may be used as a suitable carrier.

The active agent(s) or products of the invention may also find application as/in an oral formulation wherein the product is formulated in a carrier, for example selected from films, tapes, gels, microspheres, lozenges, chewing gum, dentrifices and mouthwash.

The amount of therapeutically active agent (s) that is administered and the dosage regimen for treating a disease condition with the active agent(s) or product/compositions of this invention depends on a variety of factors, including the age, weight, sex and medical condition of the subject, the severity of the disease, the route and frequency of administration, and the particular compound employed, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

The active agent(s) or products of the invention are preferably administered to the respiratory tract. Thus, the present invention also provides aerosol pharmaceutical formulations comprising the active agent(s) or product of the invention. Also provided is a nebuliser or inhaler containing the active agent(s) or product of the invention.

Additionally, the active agent(s) or products of the invention may be suited to formulation as sustained release dosage forms and the like. The formulations can be so constituted that they release the active agents, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g. stents, catheters, peritoneal dialysis tubing, draining devices and the like.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

The invention will now be described by way of Examples only with reference to the following Figures in which:

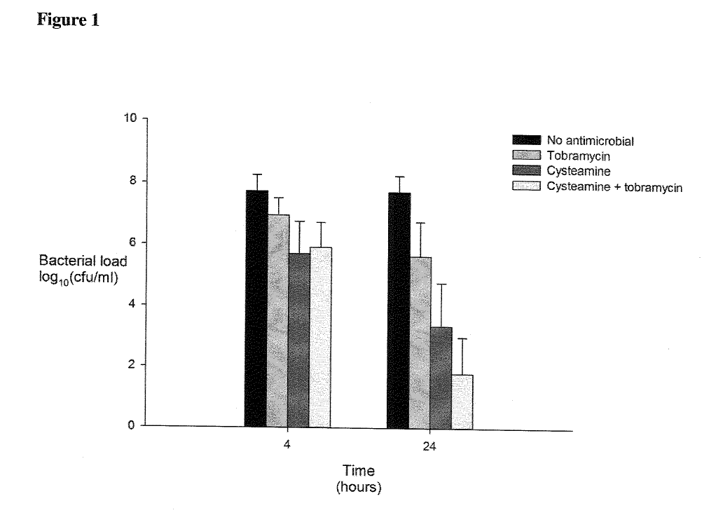

FIG. 1 is a histogram demonstrating the antimicrobial activity of tobramycin, cysteamine and combined tobramycin and cysteamine on polymicrobial load after 4 and 24 hours exposure. Bacterial load expressed as mean (95% confidence interval).

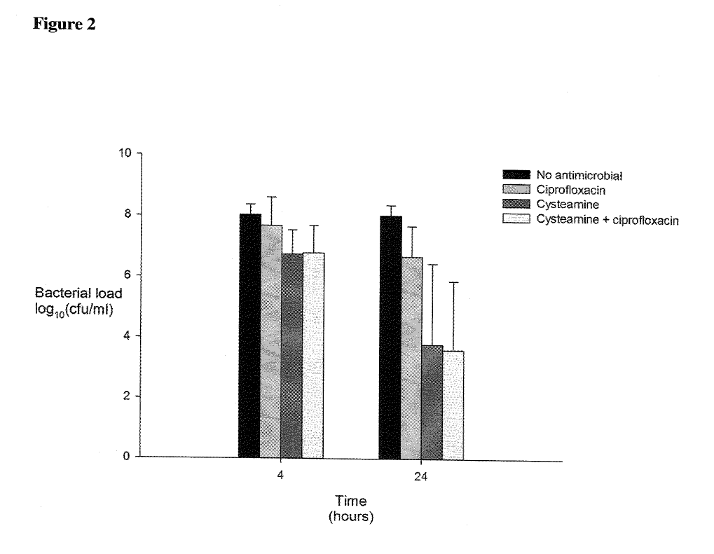

FIG. 2 is a histogram demonstrating the antimicrobial activity of ciprofloxacin, cysteamine and combined ciprofloxacin and cysteamine on polymicrobial load after 4 and 24 hours exposure. Bacterial load expressed as mean (95% confidence interval).

The following Example illustrates the invention.

### Example 1

### Methods and Materials

### Subjects

All patients with CF lung disease attending the adult CF clinic at Aberdeen Royal Infirmary (n=57) were invited to participate in a cross-sectional study investigating the antimicrobial properties of cysteamine in sputum from people with CF. Participants provided a sample of spontaneously expectorated sputum. The clinical data collected for CF participants included: age, sex, height, weight, FEV₁ and CF genotype. Also recorded were the bacterial species infecting the sputum and whether the participant was exacerbating, had a recent exacerbation (completed treatment <4 weeks previously) or was clinically stable (exacerbation >4 weeks previously). Antimicrobial therapy at the time of sampling was recorded eg azithromycin, inhaled therapies. The study received ethical approval (13/NS/0001) from the North of Scotland Research Ethics Service and all participants provided written informed consent.

### Chemicals and Growth Media

Tobramycin was purchased from Discovery Fine Chemicals (UK). All other chemicals, growth media and antibiotics were obtained from Sigma-Aldrich (UK).

### Effect of Cysteamine and Antibiotics on CF Sputum Microbial Burden: Single Exposure.

Sputum samples were processed for antimicrobial activity within 4 h of collection. To assess the antimicrobial impact of cysteamine alone and cysteamine in conjunction with antibiotics commonly used to treat infective exacerbations of CF lung disease, 0.2 ml of each sputum sample sputum was diluted ten fold in sterile phosphate buffered saline (PBS) and vortexed. Aliquots (0.2 ml) of the homogenised diluted sample were exposed to cysteamine only (1 mg/ml), [ ] antibiotic (tobramycin [0.1 mg/ml] or ciprofloxacin, [0.1 mg/ml] only, cysteamine plus antibiotic or vehicle (PBS) only, for 4 and 24 h at 37° C. Sputum samples were then serially diluted 10-fold (1×10⁻¹ to 1×10⁻⁸) and spread-plated on non-selective nutrient agar plates. Cultures were incubated at 37° C. and cfu/ml of bacteria quantified at 48 h.

### Determination of CF Sputum Microbial Burden: Multiple Exposures

To assess any impact of cysteamine on sputum microbial burden in CF at physiological concentrations feasible in vivo, multiple dosing experiments were conducted. In these assays, 0.2 ml of sputum were exposed daily (first dosing within 4 h of collection as above) to 2 μg/ml of cysteamine, or PBS as a control, for 14 days. 2 μg/ml is a level typically reported when dosing with cysteamine in patients with cystinosis. On alternate days, 10 μl of each sample was recovered and serially diluted 10-fold (1×10⁻¹ to 1×10⁻⁸) and spread-plated on non-selective nutrient agar plates. Cultures were incubated at 37° C. and cfu/ml of bacteria quantified at 48 h.

### Assessment of CF Sputum Macrorheologic Properties

Macrorheological analysis was conducted within 4 h of collection of spiutum samples. Aliquots (0.2 ml) of sputum were incubated for 1 h at 37° C. after the addition of cysteamine (1 mg/ml), PBS or DNAse (500 U/ml). The treated sputum was then transferred to the open end of a 2 ml pipette (Greiner, UK) secured vertically and allowed to descend inside the pipette under gravity. This process was filmed, and the velocity of the sputum was calculated as distance traveled over time taken in mm/s.

Antimicrobial Susceptibility of Mycobacteria abcsessus Sputum Isolates In Vitro

Mycobacterium absessus complex (MAC) was isolated from three participating patients. The susceptibility of these isolates, plus the MAC type strain M. abscessus DSMZ44196 to the antimicrobial effects of cysteamine alone and cysteamine combined with antibiotics employed in MAC eradication strategies, (amikacin, azithromycin and meropenem) was assessed by CLSI broth microdilution procedure [CLSI, 2012] and checkerboard assay respectively.

### Statistical Considerations.

FEV₁ was expressed as a percentage of predicted using GLI 2012 reference equations. Sputum microbial load expressed as colony forming units approximated to a log-normal distribution and was therefore logarithmically transformed to base 10. Microbial load after incubation with cysteamine, tobramycin, ciprofloxacin after 4 and 24 hours was modelled using two way repeated measures ANOVA with post hoc testing using Bonferroni adjustment. Analyses were performed using IBM SPSS Statistics for Windows, v22.0 (Armonk, N.Y.).

### Results

### Patient Population

Sputum samples were provided by 23 patients, their clinical characteristics are outlined in table 1.

### Antimicrobial Activity of Cysteamine Against Polymicrobial Burden in CF Sputum

The antimicrobial activity of tobramycin, cysteamine and combined tobramycin/cysteamine was tested in 23 samples and are presented in FIG. 1. Tobramycin, cysteamine and combined cysteamine/tobramycin significantly (p<0.001) reduced polymicrobial load by 1.42 (95% CI 0.92-1.92), 3.18 (2.30-4.07), and 3.86 (3.11-4.61) log₁₀ units respectively. When compared with tobramycin, cysteamine further reduced polymicrobial load by 1.76 (95% CI 0.89-2.63, p<0.001) log₁₀ units. Overall when compared with tobramycin, combined cysteamine/tobramycin further reduced polymicrobial load by 0.68 (95% CI −0.05-1.41, p=0.0⁶⁶) log₁₀ units, however after 24 hours of exposure cysteamine/tobramycin further reduced polymicrobial load by 3.85 (95% CI 2.63-5.07, p<0.001) log₁₀ units.

There was sufficient sputum to test antimicrobial activity of ciprofloxacin, cysteamine and combined ciprofloxacin/cysteamine in nine samples and the results are presented in FIG. 2. Ciprofloxacin, cysteamine and combined cysteamine/ciprofloxacin significantly reduced polymicrobial load by 0.84 (95% CI 0.29-1.39, p=0.008), 2.76 (1.32-4.20, p=0.002), and 2.86 (1.68-3.98, p<0.001) log₁₀ units respectively. When compared with ciprofloxacin, cysteamine further reduced polymicrobial load by 1.92 (95% CI 0.85-3.00, p=0.003) log₁₀ units. Overall when compared with ciprofloxacin combined cysteamine/ciprofloxacin further reduced polymicrobial load by 1.99 (95% CI 1.02-2.96, p<0.001) log₁₀ units, however combined cysteamine/ciprofloxacin did not reduce polymicrobial load over and above that achieved by cysteamine alone.

Further modelling including clinical factors demonstrated that subject CF genotype, exacerbation status or concomitant use of azithromycin or inhaled antibiotics did not appear to influence the antimicrobial effects reported above, however these analyses had less statistical power to detect associations.

Antimicrobial Susceptibility of Mycobateria abcsessus Sputum Isolates In Vitro

The Mycobacterium abscessus isolated from three of the study subjects were all sensitive to the antimicrobial effects of cysteamine (MIC range 62.5-250 μg/ml) when tested in vitro, as was the type strain DSMZ44196 (Table 2). Furthermore, cysteamine potentiated the impact of amikacin in both clinical and type strains, and azithromycin in all but one clinical strain when assessed by calculating fractional inhibitory concentration indices (FICI) which demonstrated the more than additive or synergistic potential of combined therapy. Cysteamine had concentration-dependent effects on meropenem sensitivity in the clinical isolates and type strain of M. abscessus tested, showing some concentration-specific antagonism.

TABLE 1
Clinical characteristics of participating patients.

| Age (yrs) (median, IQR) | 28 | (19-36) |
|---|---|---|
| Female (n, %) | 13 | (57%) |
| DF508/DF508 (n, %) | 16 | (70%) |
| DF508/− | 6 | (26%) |
| BMI (mean 95% CI) | 22.1 | (20.9-23.3) |
| FEV₁ % predicted (mean 95% CI) | 62% | (49-74) |
| Last exacerbation (n, %) | | |
| Acute | 9 | (39%) |
| <4 weeks | 8 | (35%) |
| >4 weeks | 6 | (26%) |
| Concomitant medication | | |
| Azithromycin (n, %) | 20 | (87%) |
| Inhaled antibiotic (n, %) | 20 | (87%) |
| Ivacaftor (n, %) | 2 | (9%) |
| Sputum culture (n, %) | | |
| Staphylococcus aureus | 4 | (17%) |
| Pseudomonas aeruginosa | 15 | (65%) |
| Burkholderia spp | 4 | (17%) |
| Stenotrophomonas maltophilia | 2 | (9%) |
| Mycobacterium abscessus | 3 | (13%) |

TABLE 2
Cysteamine is active against a panel of M. abscessus strains, and synergy with the clinically relevant antibiotics, amikacin and
azithromycin is demonstrated by chequerboard experiments and calculation of fractional inhibitory concentration index.

| | MIC₁₀₀ | | | | | | | | | |
|---|---|---|---|---|---|---|---|---|---|---|
| | Cysteamine | Meropenem | Amikacin | Azithromycin | | | | | | |
| Strain | [μg/ml] | [μg/ml] | [μg/ml] | [μg/ml] | Mero/Cys (FICI) | | Amik/Cys (FICI) | | Azi/Cys (FICI) | |
| DSM44196 | 62.5-125 | 64 | 4-8 | 1-2 | 32- | Neutral | 1-4/15.625- | Additive | 0.25/15.625 | Synergy |
| (type) | | | | | 64/62.5 | | 62.5 (0.5- | | (0.25-0.5) | |
| | | | | | (1.5-2) | | 0.75) | | | |
| MR313292 | 125-250 | 16-64 | 4 | 8 | 32- | Neutral- | <1/<15.125- | Synergy | 4-8/250 | Neutral |
| (clinical) | | | | | 64/250 | antagonistic | 31.25 | | (1.25-2) | |
| | | | | | (2-2.5) | | (0.375) | | | |
| MR27419N | 125 | 64 | 16 | 1 | 64/125 | Neutral | 4/62.5 | Additive | 0.25/<15.625 | Synergy |
| (clinical) | | | | | (2) | | (0.75) | | (0.375) | |
| MR313367D | 62.5-125 | 16-64 | 16 | 1 | 64/125 | Neutral- | 4/15.625- | Synergy | 0.25/15.625 | Synergy |
| (clinical) | | | | | (2-5) | antagonistic | 31.25 (0.5) | | (0.375-0.5) | |

All results are presented represent the mean of triplicate samples from experiments conducted three times.

1. An amino thiol compound, or pharmaceutically acceptable salt thereof, for use in the treatment and/or prevention of an infection caused by the bacterium, Mycobacterium spp., or a disease or condition associated therewith.

2. The amino thiol compound of claim 1 which is cysteamine or a derivative thereof.

3. The amino thiol compound of claim 1 wherein the Mycobacterium spp. is Mycobacterium abscessus.

4. The amino thiol compound of claim 1 wherein the infection is a biofilm infection.

5. The amino thiol compound of claim 4 wherein the infection, or disease or condition associated therewith, is selected from the group consisting of respiratory infections, infections in cystic fibrosis, chronic obstructive pulmonary disease (COPD), skin and wound infections, middle-ear infections, gastrointestinal tract infections, peritoneal membrane infections, urogenital tract infections, oral soft tissue infections, formation of dental plaque, eye infections, endocarditis and infections of indwelling medical devices.

6. The amino thiol compound of claim 5 wherein the infection is an infection associated with cystic fibrosis.

7. The amino thiol compound of claim 6 wherein the infection is a respiratory infection.

8. A pharmaceutical composition comprising an amino thiol compound, or pharmaceutically acceptable salt thereof, and a pharmaceutically acceptable carrier or diluent for use in the treatment and/or prevention of an infection caused by the bacterium, Mycobacterium spp., or a disease or condition associated therewith.

9. The amino thiol compound of claim 1, or the pharmaceutical composition of claim 8, further comprising an antibiotic.

10. A method of treating an infection caused by the bacterium, Mycobacterium spp., or disease or condition therewith associated in a patient comprising administering an effective amount of an amino thiol compound, or pharmaceutically acceptable salt thereof, to the patient.

11. A method of preventing biofilm formation in an environment comprising the step of administering an effective amount of an amino thiol compound, or pharmaceutically acceptable salt thereof, to the environment.

12. The method of claim 11 wherein the environment is infected with Mycobacterium spp.

13. The method of claim 10 wherein the environment is the mouth.

14. The method of claim 13 wherein the infection is selected from gingivitis, periodontitis and mucositis.
